(19) United States
(12) Patent Application Publication
(10) Pub. No.: US 2023/0085062 A1
(43) Pub. Date: Mar. 16, 2023

(54) GENERATING A RECOMMENDED PERIODIC HEALTHCARE PLAN

(71) Applicant: Koninklijke Philips N.V., Eindhoven (NL)

(72) Inventors: Yi Zhou, Shanghai (CN); Bin Yin, Shanghai (CN); Mengzhe Tao, Shanghai (CN); Jing Han, Shanghai (CN); Xinying Zhao, Shanghai (CN)

(21) Appl. No.: 17/940,041

(22) Filed: Sep. 8, 2022

(30) Foreign Application Priority Data
Sep. 8, 2021 (CN) PCT/CN2021/117081

(51) Int. Cl. G16H 10/60; G16H 50/30

(52) U.S. Cl. CPC G16H 10/60; G16H 50/30

(57) ABSTRACT

A mechanism for predicting or recommending how often a subject should undergo a treatment or diagnosis procedure for one or more diseases and/or conditions. Thus, the mechanism recommends a frequency for the treatment or diagnosis procedure. The recommended frequency is responsive to a risk level of the subject, which is derived from at least location information of the subject.

### FIELD OF THE INVENTION

The present invention relates to the field of healthcare, and in particular, to the generation of a healthcare plan.

### BACKGROUND OF THE INVENTION

It is increasingly common to use periodic health examinations, or check-up examinations, to assess general health and prevent future illness, rather than responding to symptoms of a patient/subject. The main aim of such examinations is to reduce morbidity and mortality by identifying modifiable risk factors and early signs of treatable disease. This form of healthcare has gained increasing attention due to at least to a shift in health landscape, namely from a reactive care approach to a proactive and preventive care approach.

There is an ongoing desire to improve periodic health examinations, specifically to make them more appropriate for a particular user and/or to ensure that potentially preventable diseases and/or conditions are identified at an early stage of development. Simultaneously, there is a desire to reduce unnecessary medical examination of a patient/subject, as excessive examinations have been shown to frustrate subjects and clinicians, and lead to subject becoming less inclined to attend a periodic health examination, as well as wasting resource.

A new approach to generating a recommended periodic healthcare plan would therefore be advantageous.

### SUMMARY OF THE INVENTION

The invention is defined by the claims.

According to examples in accordance with an aspect of the invention, there is provided a computer-implemented method of generating a recommended periodic healthcare plan for a target subject, wherein the recommended periodic healthcare plan indicates a recommended frequency for performing one or more treatment or diagnostic procedures on the target subject.

The computer-implemented method comprises: obtaining location information of the target subject, the location information indicating one or more historic locations of the target subject; processing at least the location information to predict, for each of one or more diseases or conditions, a risk level of the target subject to the said disease or condition; and generating a recommended periodic healthcare plan including, for each of the one or more diseases or conditions, a recommended periodicity for performing a treatment or diagnostic procedure for said disease or condition, wherein the recommended periodicity is responsive to at least the risk level of the target subject.

The proposed approach modifies a recommended frequency or periodicity for performing a particular treatment or diagnostic procedure on a target subject based on location information of the target subject. The subject's risk level for a particular disease/condition is determined based on at least the location information, and used to recommend a frequency at which the subject is checked or treated for a particular condition/disease.

This means that the recommended periodic healthcare is dependent upon the environment in which the subject is located, e.g. based on local rates for a particular disease/condition that thereby acts as a risk factor for that disease/condition. The proposed approach provides a more efficient periodic healthcare plan, to avoid unnecessary treatment or analysis of the subject whilst ensuring that such treatments/analysis is performed to maintain a high likelihood of catching or preventing a disease/condition. This has a direct impact on the health of the subject.

Embodiments are based on a realization that location information of a subject influence disease risk, occurrence, progression, and severity. In particular, the location information may describe variation in outcomes resulting from non-genetic or non-inherited factors, e.g. to act as a proxy for environmental and socioeconomical factors that influence risk factors for a subject.

Embodiments thereby provide a more precise estimation of the possibility of developing certain disease, a more dynamic assessment that is able to continuously monitor and assess risk changes and reduce medical expense and unwanted burden in conducting periodic treatment/diagnostic procedures.

Embodiments also recognize that, with the advent of portable electronic devices such as smartphones, location information becoming easy and effortless to measure, meaning that this invention can be easily adopted and widely used.

A periodic healthcare plan is a plan or scheme for a subject that defines how often or how frequently one or more specific tests, procedures and/or treatments should be performed on the subject. The treatment procedure may be a preventative treatment procedure, such as a vaccination.

In some examples, the step of processing at least the location information to predict a risk level for each of one or more diseases or conditions comprises, for each disease or condition: obtaining a first disease/condition rate for other subjects in a historic location of the target subject indicated in the location information; and using at least the first disease/condition rate to predict a risk level for the target subject to the said disease or condition.

This approach thereby uses location-based rates of a disease/condition in the prediction of the risk level for the subject. This provides an accurate, evidence-based and reliable approach for assessing a risk level of the subject.

Embodiments may further comprise obtaining target subject data comprising information about one or more characteristics of the target subject, wherein the processing at least the location information comprises processing at least the location information and the target subject data to predict, for each of one or more diseases or conditions, the risk level.

Other characteristics of the target subject are known to have an influence on likelihood of a disease or condition. For instance, age, biological sex and family history of a particular disease have been identified as being key risk factors in predicting whether or not a subject is likely to get the disease.

By taking such characteristics into account, a yet further accurate indication of a risk level for a subject can be generated.

In at least one example, the target subject data comprises an age and/or biological sex of the target subject, wherein the processing at least the location information comprises processing at least the location information and the age and/or biological sex of the target subject data to predict, for each of one or more diseases or conditions, the risk level.

In some examples, the step of processing the location information to predict a risk level for each of one or more diseases or conditions comprises, for each disease or condition: obtaining a second disease/condition rate for other subjects in a same age group and/or biological sex as the target subject; and using at least the second disease/condition rate to predict a risk level for the target subject to the said disease or condition.

This approach thereby uses age or biological sex based rates of a disease/condition in the prediction of the risk level for the subject. This improves an accuracy, reliability and trustworthiness of the approach for assessing a risk level of the subject.

Optionally, the step of processing the location information to predict a risk level for each of one or more diseases or conditions comprises, for each disease or condition: combining, and optionally weighting, the first and second disease rates to produce the predicted risk level for the target subject to the said disease or condition.

Methods may further comprise obtaining genomic data of the target subject, comprising information on one or more genetic factors of the target subject, wherein the processing at least the location information comprises processing at least the location information and the genomic data to predict, for each of one or more diseases or conditions, the risk level.

The genomic data may comprise, for instance, the result of one or more genomic tests performed on the target subject, e.g. one or more genomic markers or genomic factors of the target subject. Genomic factors have been shown to influence a risk level for a subject of obtaining a disease/condition. Using genomic data to generate a risk level thereby further increases an accuracy and reliability of the determined risk level.

The step of processing the location information to predict a risk level for each of one or more diseases or conditions preferably comprises, for each disease or condition: processing the genomic data to identify any genomic factors that influence a risk or rate of the disease or condition; and using the identified genomic factors to predict a risk level for the target subject to the said disease or condition.

The one or more diseases or conditions may comprise only: non-infectious diseases or conditions; and/or long-term, degenerative and/or chronic diseases or conditions.

The one or more treatment or diagnostic procedures may comprise at least one preventative treatment procedure, such as at least one vaccination. In some examples, the one or more treatment or diagnostic procedures comprises at least one disease screening procedure.

Embodiments may further comprise providing a visual representation of the recommended periodic healthcare plan. In this way, the subject, clinician, caregiver and/or other interested party (e.g. a family member) can be advised of the recommended periodic healthcare plan to ensure that the periodic healthcare plan is followed.

In some examples, the recommended periodic healthcare plan may be stored and could be used, for instance, to generate reminders for the subject or other interested party to schedule a treatment/diagnosis procedure.

There is also proposed a computer program product comprising computer program code means which, when executed on a computing device having a processing system, cause the processing system to perform all of the steps of any herein described method.

There is also proposed a processing system configured to generate a recommended periodic healthcare plan for a target subject, wherein the recommended periodic healthcare plan indicates a recommended frequency for performing one or more treatment or diagnostic procedures on the target subject.

The processing system is configured to: obtain location information of the target subject, the location information indicating one or more historic locations of the target subject; process at least the location information to predict, for each of one or more diseases or conditions, a risk level of the target subject to the said disease or condition; and generate a recommended periodic healthcare plan including, for each of the one or more diseases or conditions, a recommended periodicity for performing a treatment or diagnostic procedure for said disease or condition, wherein the recommended periodicity is responsive to at least the risk level of the target subject.

The skilled person would be readily capable of modifying any herein described processing system to perform the steps of any herein described method, and vice versa.

There is also proposed a system comprising: the processing system herein described; and a user interface configured to display a visual representation of the recommended periodic healthcare plan.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 provides an overview of an approach of the disclosure;

FIG. 2 is a flowchart illustrating a method according to an embodiment;

FIG. 3 illustrates a processing system according to an embodiment; and

FIG. 4 illustrates a system according to an embodiment.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a mechanism for predicting or recommending how often a subject should undergo a treatment or diagnosis procedure for one or more diseases and/or conditions. Thus, the mechanism recommends a frequency for the treatment or diagnosis procedure. The recommended frequency is responsive to a risk level of the subject, which is derived from at least location information of the subject.

The present invention recognizes that subjects in certain geographic locations would benefit from more frequency screening or preventative treatment procedures, due to increased risk of certain diseases/conditions in those areas. The proposed approach assesses a risk level of the subject, based on at least their location, and recommends a frequency of treatment.

Disclosed embodiments thereby provide information useful for appropriate medical treatment and/or analysis of the subject, which provides a clinician and/or subject with useful information and guidance that reduces the chance that a disease/condition will be overlooked. In particular, excessive screening and/or treatment can lead to wasted resources, disinclination of a subject to attend screening/treatment, overburdening of available healthcare resource and a false sense of security. Under screening or treatments can lead to missed identification or treatment of a disease/condition. There is a direct link between frequency of treatment/diagnosis procedures and health outcomes of the subject. By recommending a treatment/diagnosis procedural frequency based on location information, a balance between these two possibilities is achieved, thereby improving the health of the subject.

Embodiments may be employed in any medical or veterinary environment in which periodic examinations or treatments are recommended, e.g. for the purposes of preventive medical or veterinary practice(s).

In the context of the present disclosure, a subject may be a human or an animal under the care and/or responsibility of a clinician. The term “patient” may be interchangeable with the term “subject”.

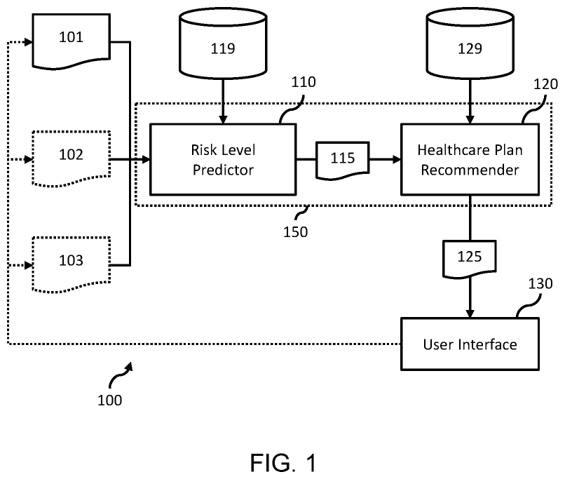

FIG. 1 conceptually illustrates an approach adopted by embodiments of this disclosure, and in particular, a dataflow 100 of data that is processed to generate a recommended periodic healthcare plan for a target subject.

A risk level predictor 110 determines/predicts one or more risk levels 115 of the target subject (to a respective one or more diseases or conditions) based on at least location information 101 of the target subject. The location information indicates one or more historic locations of the target subject. A different risk level may be generated for different diseases and/or conditions.

The location information 101 of the target subject may be obtained, for example, from GPS tracking information of the subject (e.g. from a mobile phone of the subject or the like). Other example approaches for obtaining or supplementing location information 101 will be apparent to the skilled person, e.g. based on a recorded home address of the subject and/or based on a user input defining a location of the subject.

The risk level predictor may use a risk factor library 119 in order to predict the risk level(s). For instance, the risk factor library may indicate a relationship or mapping between particular locations and an influence on the risk level. The historic location(s) indicated in the location information may then be used in the determination or prediction of the risk level, by determining the influence of each historic location on the risk level of the subject. More detailed examples will be provided later in this disclosure.

The risk factor library 119 may effectively link risk factors (e.g. locations) to the development and progress of certain diseases and/or conditions, to facilitate determination of a risk level from known risk factors of the subject (including at least the location information).

The risk level predictor 110 may use a proportional hazards model (e.g. a Cox model) in the determination of the one or more risk levels.

In some embodiments, the risk level predictor uses other information or risk factors of the subject in their assessment or prediction of the risk level of the subject. This approach recognizes that other factors may affect or influence a likely risk of a subject to a particular disease, i.e. are risk factors, such that an improved accuracy in identifying a risk level can be achieved.

Examples of other information include personal/identifying details or characteristics 102 of the subject (e.g. age, biological sex, family history and so on); genetic or genomic information 103 and/or other risk factors (e.g. derived from genetic material of the subject) and so on. The risk level predictor 110 may process the available risk factors 101-103, including at least the location information 101, in order to determine a risk level of the subject.

A healthcare plan recommender 120 then processes the determined risk level(s) 115 of the target subject to determine a recommended healthcare plan 125 for the subject, which includes at least a recommended periodicity/frequency for performing a treatment or diagnostic procedure for each said disease or condition (of the risk levels). In this way, the recommended periodicity is responsive to at least the risk level(s) of the target subject.

Generally, a periodicity may indicate a recommended frequency at which the treatment/diagnostic procedure should be taken (e.g. annually, biannually, monthly, every X years (where X is a real number) and so on).

It will be appreciated that the recommended periodicity may be to never have a particular treatment or diagnostic procedure (e.g. an infinite periodicity). Thus, the approach may recommended that some treatment or diagnostic procedures are not performed. This may be responsible, for instance, to a risk level for the target subject breaching some threshold and/or falling below a threshold (e.g. the risk level is so low that performing a treatment/diagnostic procedure is more likely to cause harm then identify/solve a pathology).

However, in some examples, the recommended periodicity may always be finite. This reduces a chance that a certain pathology will be missed/overlooked and/or not treated, by ensuring a minimum frequency of checking.

The healthcare plan recommender may use a recommendation library 129 to generate the recommended healthcare plan. The recommendation library may link or map a risk level of the subject to a particular disease/condition to a recommended periodicity in performing a treatment/diagnosis procedure (e.g. a preventative treatment or a screening process) for that particular disease/conditions.

It will be apparent that the recommendation library may also link a particular disease/condition to one or more treatment/diagnosis procedures. In this way, the treatment/diagnosis procedures to perform may be defined in the recommendation library.

In some examples, the healthcare plan recommender 120 may use other information to generate the recommended healthcare plan. For instance, the healthcare plan recommender 120 may further use personal/identifying details 102 (as well as the risk level) in order to generate the recommended healthcare plan.

This approach recognizes that different periodicities may be recommended for different subject groups (e.g. different age groups or different biological sexes). For instance, a person who is at potentially high risk for prostate cancer (based on their location) is unlikely to suffer from prostate cancer until they reach a certain age.

The recommended healthcare plan may be presented to the subject and/or a clinician at a user interface 130. Thus, the healthcare plan recommender 120 may pass the recommended healthcare plan to the user interface 130, which then provides a user-perceptible output representing the recommended healthcare plan. Examples of suitable user-perceptible outputs include visual and/or audio outputs.

The recommended healthcare plan may be responsive to historic treatments and/or diagnostic procedures performed for the subject, e.g. to recommend a next occasion for performing a treatment/diagnostic procedure based upon a time at which a last treatment/diagnostic procedure was performed.

The user interface 130 may also be used to provide the location information 101 and/or information on other risk factors. For instance, the subject or clinician may be able to input the location information and/or information on other risk factors into the user interface 130.

The risk level predictor 110 and the healthcare plan recommender 120 may form modules of a processing system 150.

The one or more diseases or conditions may include non-infectious diseases and/or conditions and/or long-term, degenerative and/or chronic diseases or conditions. This approach recognizes that at least diagnostic processes that are performed periodically are usually used to pre-emptively identify long term conditions (such as cancer, dementia such as Alzheimer's disease, Parkinson's disease, multiple sclerosis and so on). Location of a subject is particularly influential on long term or non-infectious diseases, such that the proposed approach is particularly advantageous in such examples.

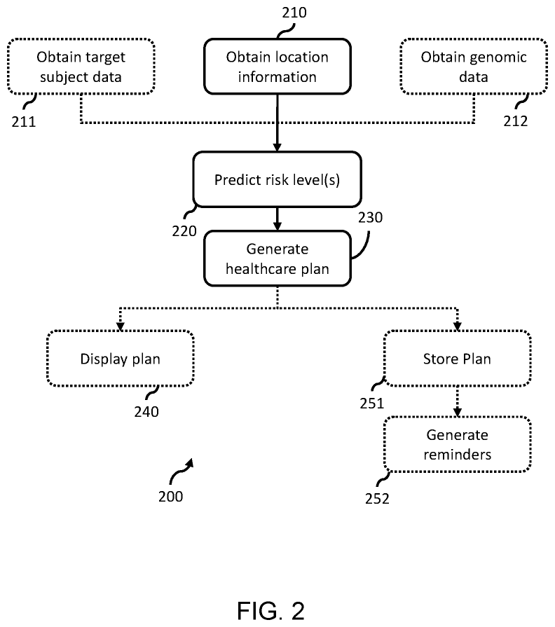

FIG. 2 illustrates a method 200 according to an embodiment. The method 200 may be carried out by a processing system. The method is configured for generating a recommended periodic healthcare plan for a target subject. The recommended periodic healthcare plan indicates a recommended frequency or periodicity for performing one or more treatment or diagnostic procedures on the target subject.

The method 200 comprises a step 210 of obtaining location information of the target subject, the location information indicating one or more historic locations of the target subject.

The method also comprises a step 220 of processing at least the location information to predict, for each of one or more diseases or conditions, a risk level of the target subject to the said disease or condition.

The method also comprises a step 230 of generating a recommended periodic healthcare plan including, for each of the one or more diseases or conditions, a recommended periodicity for performing a treatment or diagnostic procedure for said disease or condition, wherein the recommended periodicity is responsive to at least the risk level of the target subject. In particular examples, the greater the risk of the subject, the smaller the recommended periodicity between recommended treatment or diagnostic procedures (i.e. the more frequently the subject is examined/treated).

Step 220 may comprise obtaining a first disease/condition rate for other subjects in a historic location of the target subject indicated in the location information; and using at least the first disease/condition rate to predict a risk level for the target subject to the said disease or condition. In this way, a rate of a disease for subjects in a same geographic area as the target subject is used to define the risk level for that disease.

The first rate may be defined in publically available data about the disease/condition rate, e.g. as stored in a database or risk factor library. The risk factor library may further define a relationship of mapping between a rate and risk level to a subject.

In some examples, if the location information indicates more than one historic location, step 220 may comprise obtaining a rate for each indicated location in the location information. A combined rate may then be generated by combining the obtained rates, e.g. by averaging the obtained rates.

In some examples, the combined rate is generated by performing a weighted average of the obtained rates. The weights applied to each obtained rate may be responsive to an amount of time spent (by the subject) in the historic location associated with the rate. Thus, the historic information may further indicate a length of time spent by the subject in each historic location, e.g. by using timestamps to indicate a change of location or the like.

In some examples, step 220 may comprise obtaining environmental risk factors (e.g. air pollution level) based on the indicated historic location(s) of the subject. These environmental risk factors may be processed in the generation/prediction of the risk level for the subject.

In this way, a risk level can be generated based on an environmental profile, which effectively represents a combination of environmental risk factors (e.g. air pollution level) and/or regional disease patterns that can be identified from public databases.

As previously explained, other characteristics of the subject and/or genomic information of the subject may also be used in the determination of the risk level of the subject. For instance, age and biological sex are significant influences on the risk level to a particular disease or condition. Similarly, certain genes or genomic sequences may indicate a greater predisposition to certain diseases or conditions.

Thus, the method 200 may comprise obtaining, in a step 211, target subject data comprising information about one or more characteristics of the target subject. Step 220 may comprise processing at least the location information and the target subject data to predict, for each of one or more diseases or conditions, the risk level.

Similarly, the method 200 may further comprise obtaining in a step 211 genomic data of the target subject, comprising information on one or more genetic factors of the target subject. Step 220 may comprise processing at least the location information and the genomic data to predict, for each of one or more diseases or conditions, the risk level.

Of course, a combination of the location information, the target subject data and the genomic data of the target subject may be processed in step 220. This may be performed by, for instance, determining a rate of the disease for subjects sharing similar locations, subject data and/or genomic data of the target subject, and using the identified rates to predict the risk level.

The determined risk level in step 220 may be binary, categorical or numeric.

As one example, the determined risk level may indicate whether the subject is at a “normal” or “high” risk of the particular disease/condition. As another example, the determined risk level may indicate whether the subject is at a “low”, “normal” or “high” risk of the particular disease/condition. As yet another example, the determined risk level may indicate a risk on a predetermined scale (e.g. 0 to 1, 0 to 1, 1 to 10, 0 to 100 or 1 to 100).

One approach to generating a numeric risk level may be to perform a weighted sum. Each characteristic of the subject (e.g. location, age, biological sex, presence or absence of a certain gene) is assigned a value which is then weighted. The weighted values may then be summed to determine the risk level.

The location may be assigned a value based on rate of the disease/condition for other subjects in the same location. Similarly, an age or biological sex of the subject may be assigned a value based on the rate of the disease/condition for the subjects of a similar age and/or same biological sex. As yet another example, a presence or absence of a certain gene or mutation of a gene may be assigned a value based on the rate of the disease/condition for subjects sharing the same presence/absence of the gene or gene mutation.

Another example of generating a numeric risk level is to use a look-up table to cross-reference the available characteristics of a subject with a rate of the disease/condition.

Table 1 illustrates one example of how a number or rate of cases for a particular disease (here: colorectal cancer) differs depending upon area or location. This table indicates an estimated number of cases per area. Together with appropriate population data for these areas, this information can be used to derive a rate of cases per area. This rate information can be used to predict (for a subject of a particular biological sex in a particular area) a likely rate of the condition or disease.

TABLE 1
Number of Colorectal Cancer Cases in China

Biological Sex

| Area of China | Male | Female |
|---|---|---|
| North | 22.1 | 16.7 |
| Northeast | 24.5 | 16.9 |
| East | 70.5 | 55.1 |
| Central | 32.7 | 25.8 |
| South | 28.8 | 22.0 |
| Southwest | 26.5 | 16.4 |
| Northwest | 10.6 | 7.7 |

In yet another example, generating a numeric risk level may comprise processing location information (and optionally other risk factors) using an appropriately trained machine-learning method configured to generate the risk level.

In some examples, a binary or categorical indicator of risk may be calculated by determining a numeric risk level and assigning a binary value or category based on the numeric risk level—e.g. depending upon a range of values into which the numeric risk value falls.

As another example, an appropriately trained machine-leaning method may be used to process the location information (and optionally other risk factors) to generate the binary/categorical risk levels.

Step 230 may be performed using a set of mappings or rules that defines (for particular risk levels) a recommended periodicity for performing a particular treatment or diagnostic procedure based on the risk level. In some examples other characteristics of the subject (such as age or gender) may be used (as well as the risk level) to generate the recommended frequency.

As a working example, consider a scenario where the disease/condition is colorectal cancer and the treatment/diagnostic procedure is a colonoscopy for screening for colorectal cancer.

In this scenario, a subject between the ages of 45 and 75 years old with a “normal” risk level for colorectal cancer may be recommended to have a colonoscopy every 10 years, whereas an otherwise identical subject with a “high” risk level for colorectal cancer may be recommended to have a colonoscopy every 1 to 5 years.

In this same scenario, a subject younger than 45 years old with a “normal” risk level for colorectal cancer may be recommended to not have a colonoscopy, whereas an otherwise identical subject with a “high” risk level for colorectal cancer may be recommended to have a colonoscopy every 10 years.

In this way, it is apparent how the determined risk level of the subject (which is responsive to location information of the subject) can be used to recommend a periodicity or frequency of performing a treatment/diagnostic procedure.

The skilled person will appreciate that the above-described approach also applies to other forms of disease/condition and/or treatment/diagnostic procedure, such as screening for breast cancer (e.g., mammography), lung cancer (e.g., low-dose CT scan), dental review, pneumococcal immunization, etc.

The recommended periodic healthcare plan generated in step 230 may contain any recommended periodicities or frequencies generated using this approach, alongside an indicator of the corresponding disease/condition and/or treatment/diagnostic procedure.

The method 200 may further comprise a step 240 of providing a user-perceptible representation (e.g. a visual representation and/or audio representation) of the recommended periodic healthcare plan. This may be provided at a user interface, such as a display and/or audio output device.

Optionally, the method 200 further comprises a step 251 of storing the recommended periodic healthcare plan.

In some examples, the method further comprises a step 252 of using the stored recommended periodic healthcare plan to generate (user-perceptible) reminders for the subject or other interested party to schedule a treatment/diagnosis procedure. Step 252 may comprise determining a difference between a time since last performance of the treatment/diagnosis procedure (on the subject) and the recommended periodicity for performing said treatment/diagnosis procedure. Step 252 may trigger the generation of a user-perceptible alert responsive to the time since last performance of the treatment/diagnosis procedure exceeding the recommended periodicity for performing said treatment/diagnosis procedure.

The time since last performance of the treatment/diagnosis procedure may be determined from a (electronic) medical record of the subject, e.g. recording treatment/diagnosis procedures performed on the subject. In other examples, an input at a user interface may be used to identify the time of a last procedure (e.g. by a user inputting when a procedure is performed).

This step reduces a likelihood that the subject will miss a recommended treatment/diagnosis procedure.

Another illustrative example of generating a recommended periodic healthcare plan for a subject is hereafter described. Reference will be made to the dataflow of FIG. 1 for the sake of improved understanding.

In this working example, a periodic healthcare plan for cardio-vascular disease is generated for three separate subjects, namely: Subject A, Subject B and Subject C. The periodic healthcare plan provides a recommended periodicity (i.e. frequency) for performing certain screening tests for identifying cardio-vascular disease (or signs/symptoms associated with the same). These screening tests are tests for blood pressure, lipid profile, body weight, and blood glucose.

The risk level predictor 110 determines/predicts cardiovascular diseases levels 115 of the target subject based on at least location information 101 of the target subject. For three subjects who live in different locations, the location information 101 of the target subject may be obtained from GPS tracking information from a mobile phone (or other portable device) of the subject. Alternatively, the location information may be manually input (e.g. by the subject).

Subject A is identified as living in location A. Subject B is identified as living in location B₂, having moved from location B₁ three years ago. Subject C is identified as living in location C.

The risk factor library 119 indicates a relationship or mapping between a particular location and an influence on the risk level. By way of example, the location may be used to identify a region in which the subject is located, and a risk associated with the region can be identified. The regional risk level can be grouped into categories according to their characteristics.

For example, European countries can be grouped into four risk regions according to recently reported WHO age- and sex-standardized overall CVD mortality rates per 100,000 population. The four groupings can be low risk (<100 CVD deaths per 100,000), moderate risk (100 to <150 CVD deaths per 100,000), high risk (150 to <300 CVD deaths per 100,000), and very high risk (≥300 CVD deaths per 100,000).

Examples of other information that could be processed in determining a risk level or to control the recommended periodicity include personal/identifying details or characteristics 102 of the subject (e.g., age, biological sex, family history and so on); genetic or genomic information 103 and/or risk factors (e.g., derived from genetic material of the subject) and so on.

For this working example, the relevant personal/identifying details or characteristics 102 of the subject (e.g., age, biological sex, family history and so on) input by the individuals are as follows:

Subject A: Female, 58 years old;

Subject B: Male, 57 years old; and

Subject C: Male, 42 years old, with family history of CVD events

For this working example, the genetic or genomic information 103 and/or risk factors (e.g., derived from genetic material of the subject) reported by the individuals or input from third party's assessment are:

Subject A: NA (or none known);

Subject B: NA (or none known); and

Subject C: Variants in the LPL region

The risk level predictor 110 processes the available risk factors 101-103, including at least the location information, in order to determine a risk level of the subject. One approach is to use a proportional hazards model, such as a Cox model, with time-dependent covariates (e.g. to represent the time spent in a particular location), to predict the 10 year risk score for cardiovascular disease based on the available risk factors of the subject. The structure of the Cox model is set out below:

λ(t|Z(t))=λ₀(t)exp{β′Z(t)} (1)

In this example, the fixed covariates include: age of the subject (age at the time of generating plan), gender of the subject, family history of the subject (family history of cardiovascular events), and genetic information of the subject. The time-varying covariates: include the location (the time spent in one location and its risk index level). The skilled person would readily appreciate how different covariates may be associated with different risk levels, and how this can be incorporated into a proportional hazards model.

A different cardiovascular disease risk level 115 is then generated for each subject. In this working example, the determined risk level(s) 115 of each subject A-C is as follows:

Subject A: Female, 58 years old, cardiovascular risk=7%

Subject B: Male, 57 years old, cardiovascular risk=12%

Subject C Male, 42 years old, cardiovascular risk=25%

The healthcare plan recommender 120 then uses a recommendation library 129 to generate the recommended healthcare plan.

Screening for blood pressure and body weight is recommended every year. Screening for blood glucose is recommended every three years. Screening for lipid disorders should be repeated every five years for low-risk patients (ten-year cardiovascular risk <10%) and every two or five years for intermediate-risk patients (ten-year cardiovascular risk 10%-20%). Lipid-measurement should be repeated more frequently based on the clinical situation for patients with a high or very high risk (ten-year cardiovascular risk >20%).

Table 2 illustrates a recommended periodicity or frequency for performing lipid-measurements for different risk levels, ages and genders. This demonstrates an example of how predicted risk levels can be processed (along with (optionally) other information about the subject) in order to define a recommended periodicity for a particular treatment or diagnostic procedures.

For the sake of conciseness, other similar mapping or tables for other treatment or diagnostic procedures have not been included, but the skilled person would appreciate how they may be similarly structured.

TABLE 2
Recommended Periodicity for lipid-measurements

| CVD | Low-risk | | Intermediate-risk | | High-risk | |
|---|---|---|---|---|---|---|
| risk | (<10%) | | (10%-20%) | | (>20%) | |
| Age | Female | Male | Female | Male | Female | Male |
| <40 | N/A | N/A | N/A | every | every | every two |
| | | | | five | five | to five |
| | | | | years | years | years |
| 40-50 | N/A | every | every | every two | every two | every |
| | | five | five | to five | to five | year |
| | | years | years | years | years | |
| >50 | every | every | every two | every two | every | every |
| | five | five | to five | to five | year | year |
| | years | years | years | years | | |

Continuing with the working example, the healthcare plan recommender 120 processes the determined risk level(s) 115 of the target subjects to determine a recommended healthcare plan 125 for each subject. In this working example, each subject is recommended the following periodicities (e.g. making use of Table 2 and the generally recommended periodicities).

Subject A: blood pressure and body weight: every year; blood glucose: every three years; lipid profile: every five years.

Subject B: blood pressure and body weight: every year; blood glucose: every three years; lipid profile: every two to five years.

Subject C: blood pressure, body weight and lipid profile: every year; blood glucose: every three years.

It will be appreciated from the foregoing (e.g., Table 2) that some recommended healthcare plans may indicate that certain diagnostic or treatment procedures do not need to be performed (i.e. should be performed at an infinite periodicity or zero frequency). Thus, procedures/treatments may be included or excluded/removed from the healthcare plan based on at least the risk level(s).

The recommended healthcare plan may be presented to the subject and/or a clinician at a user interface 130. The recommended healthcare plan may be responsive to historic treatments and/or diagnostic procedures performed for the subject. For instance:

Subject A: subject A has been screened for lipid disorders and blood glucose level two years ago. The recommendation for subject A is to have the annual check for blood pressure and body weight, and to have a blood glucose test next year, a lipid profile test three years later.

Subject B: subject B hasn't been screened for lipid disorders and blood glucose level before. It is recommended to initiate the screening this year, in combine with blood pressure and body weight, and have follow up test every three years.

Subject C: no information about previous check-ups. Subject C is recommended to have blood pressure, body weight, and lipid profile tested every year, and blood glucose every three years.

Whilst the above examples have been described in the context of recommending a periodicity for a diagnostic screening (e.g. for colorectal cancer or cardiovascular disease), embodiments are also useful for generating a recommended periodicity between (preventative) treatments. For instance, if a region containing a historic location indicated in the location information has a particularly high risk of a certain disease, then a preventative treatment (such as a vaccination) for that disease may be recommended to be taken more frequently to reduce the risk of contracting that disease.

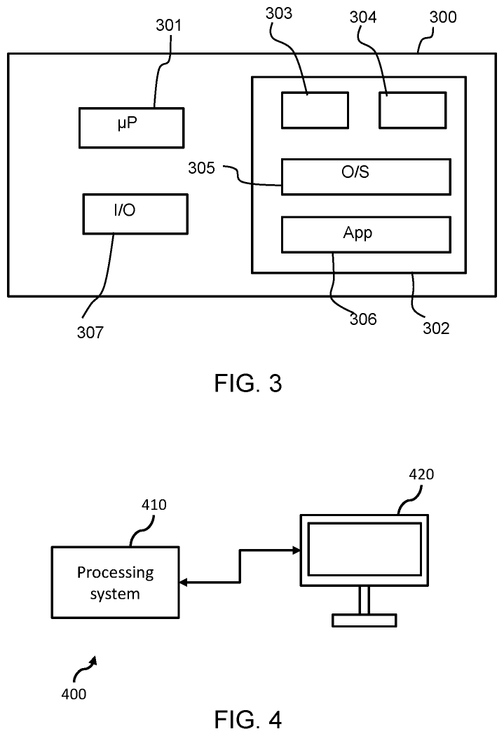

By way of further example, FIG. 3 illustrates an example of a processing system 300 within which one or more parts of an embodiment may be employed. Various operations discussed above may utilize the capabilities of the processing system 300. For example, one or more parts of a system for generated a recommended periodic healthcare plan for a target subject may be incorporated in any element, module, application, and/or component discussed herein. In this regard, it is to be understood that system functional blocks can run on a single computer or may be distributed over several computers and locations (e.g. connected via internet).

The processing system 300 includes, but is not limited to, PCs, workstations, smartphones, laptops, PDAs, palm devices, servers, storages, and the like. Generally, in terms of hardware architecture, the processing system 300 may include one or more processors 301, memory 302, and one or more I/O devices 307 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 301 is a hardware device for executing software that can be stored in the memory 302. The processor 301 can be virtually any custom made or commercially available processor, a central processing unit (CPU), a digital signal processor (DSP), or an auxiliary processor among several processors associated with the processing system 300, and the processor 301 may be a semiconductor based microprocessor (in the form of a microchip) or a microprocessor.

The memory 302 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 302 may incorporate electronic, magnetic, optical, and/or other types of storage media. Note that the memory 302 can have a distributed architecture, where various components are situated remote from one another, but can be accessed by the processor 301.

The software in the memory 302 may include one or more separate programs, each of which comprises an ordered listing of executable instructions for implementing logical functions. The software in the memory 302 includes a suitable operating system (O/S) 305, compiler 304, source code 303, and one or more applications 306 in accordance with exemplary embodiments. As illustrated, the application 306 comprises numerous functional components for implementing the features and operations of the exemplary embodiments. The application 306 of the processing system 300 may represent various applications, computational units, logic, functional units, processes, operations, virtual entities, and/or modules in accordance with exemplary embodiments, but the application 306 is not meant to be a limitation.

The operating system 305 controls the execution of other computer programs, and provides scheduling, input-output control, file and data management, memory management, and communication control and related services. It is contemplated by the inventors that the application 306 for implementing exemplary embodiments may be applicable on all commercially available operating systems.

Application 306 may be a source program, executable program (object code), script, or any other entity comprising a set of instructions to be performed. When a source program, then the program is usually translated via a compiler (such as the compiler 304), assembler, interpreter, or the like, which may or may not be included within the memory 302, so as to operate properly in connection with the O/S 305. Furthermore, the application 306 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 307 may include input devices such as, for example but not limited to, a mouse, keyboard, scanner, microphone, camera, etc. Furthermore, the I/O devices 307 may also include output devices, for example but not limited to a printer, display, etc. Finally, the I/O devices 307 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 307 also include components for communicating over various networks, such as the Internet or intranet.

If the processing system 300 is a PC, workstation, intelligent device or the like, the software in the memory 302 may further include a basic input output system (BIOS) (omitted for simplicity). The BIOS is a set of essential software routines that initialize and test hardware at startup, start the O/S 305, and support the transfer of data among the hardware devices. The BIOS is stored in some type of read-only-memory, such as ROM, PROM, EPROM, EEPROM or the like, so that the BIOS can be executed when the processing system 300 is activated.

When the processing system 300 is in operation, the processor 301 is configured to execute software stored within the memory 302, to communicate data to and from the memory 302, and to generally control operations of the processing system 300 pursuant to the software. The application 306 and the O/S 305 are read, in whole or in part, by the processor 301, perhaps buffered within the processor 301, and then executed.

When the application 306 is implemented in software it should be noted that the application 306 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 306 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

FIG. 4 illustrates a system 400 according to an embodiment. The system 400 comprises a processing system 410 and a user interface 420.

The processing system 410 may be configured to perform any herein described method to generate a periodic healthcare plan. The processing system may be embodied as described with reference to FIG. 3.

The user interface may be configured to generate a user-perceptible output, e.g. a visual display, of the recommended periodic healthcare plan generated by the processing system. The processing system 410 may control the operation of the user interface.

The user interface 420 may also be used to input information on the subject, including location information and optionally other risk factors.

Optionally, the user interface may also be used to obtain information on treatment/diagnosis procedures performed on the subject, e.g. to control the generation of alerts or reminders for performing a further treatment/diagnosis procedure based on a recommended periodicity.

Embodiments may make use of a machine-learning algorithm to generate or predict a risk levels based on one or more risk factors of a subject (which includes at least location information).

A machine-learning algorithm is any self-training algorithm that processes input data in order to produce or predict output data. Here, the input data comprises one or more risk factors of a subject (including at least location information) and the output data comprises a predicted risk level of the subject to a particular disease or condition.

In some examples, the machine-learning algorithm may be trained to predict a probability of whether a subject having the risk factors (of the input data) suffers from the particular disease/condition.

Suitable machine-learning algorithms for being employed in the present invention will be apparent to the skilled person. Examples of suitable machine-learning algorithms include decision tree algorithms and artificial neural networks. Other machine-learning algorithms such as logistic regression, support vector machines or Naive Bayesian models are suitable alternatives.

The structure of an artificial neural network (or, simply, neural network) is inspired by the human brain. Neural networks are comprised of layers, each layer comprising a plurality of neurons. Each neuron comprises a mathematical operation. In particular, each neuron may comprise a different weighted combination of a single type of transformation (e.g. the same type of transformation, sigmoid etc. but with different weightings). In the process of processing input data, the mathematical operation of each neuron is performed on the input data to produce a numerical output, and the outputs of each layer in the neural network are fed into the next layer sequentially. The final layer provides the output.

Methods of training a machine-learning algorithm are well known. Typically, such methods comprise obtaining a training dataset, comprising training input data entries and corresponding training output data entries. An initialized machine-learning algorithm is applied to each input data entry to generate predicted output data entries. An error between the predicted output data entries and corresponding training output data entries is used to modify the machine-learning algorithm. This process can be repeated until the error converges, and the predicted output data entries are sufficiently similar (e.g. ±1%) to the training output data entries. This is commonly known as a supervised learning technique.

For example, where the machine-learning algorithm is formed from a neural network, (weightings of) the mathematical operation of each neuron may be modified until the error converges. Known methods of modifying a neural network include gradient descent, backpropagation algorithms and so on.

The training input data entries correspond to example (values of) risk factors. The training output data entries correspond to presence or occurrence of the disease/condition in the subject.

A machine-learning method may be configured to generate or output a probability or confidence that processed risk factors are associated with a subject suffering from a condition. The probability/confidence may be used as a numeric risk value. The probability/confidence may undergo binning (e.g. compared to one or more thresholds or ranges) to produce a binary/categorical risk value.

It will be understood that disclosed methods are preferably computer-implemented methods. As such, there is also proposed the concept of a computer program comprising code means for implementing any described method when said program is run on a processing system, such as a computer. Thus, different portions, lines or blocks of code of a computer program according to an embodiment may be executed by a processing system or computer to perform any herein described method. In some alternative implementations, the functions noted in the block diagram(s) or flow chart(s) may occur out of the order noted in the figures. For example, two blocks shown in succession may, in fact, be executed substantially concurrently, or the blocks may sometimes be executed in the reverse order, depending upon the functionality involved.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. If a computer program is discussed above, it may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted the term “adapted to” is intended to be equivalent to the term “configured to”. Any reference signs in the claims should not be construed as limiting the scope.

1. A computer-implemented method of generating a recommended periodic healthcare plan for a target subject, wherein the recommended periodic healthcare plan indicates a recommended frequency for performing one or more treatment or diagnostic procedures on the target subject, the computer-implemented method comprising:
obtaining location information of the target subject, the location information indicating one or more historic locations of the target subject;
processing at least the location information to predict, for each of one or more diseases or conditions, a risk level of the target subject to the said disease or condition; and
generating a recommended periodic healthcare plan including, for each of the one or more diseases or conditions, a recommended periodicity for performing a treatment or diagnostic procedure for said disease or condition, wherein the recommended periodicity is responsive to at least the risk level of the target subject.

2. The computer-implemented method of claim 1, wherein the step of processing at least the location information to predict a risk level for each of one or more diseases or conditions comprises, for each disease or condition:
obtaining a first disease/condition rate for other subjects in a historic location of the target subject indicated in the location information; and
using at least the first disease/condition rate to predict a risk level for the target subject to the said disease or condition.

3. The computer-implemented method of claim 1, further comprising obtaining target subject data comprising information about one or more characteristics of the target subject,
wherein the processing at least the location information comprises processing at least the location information and the target subject data to predict, for each of one or more diseases or conditions, the risk level.

4. The computer-implemented method of claim 3, wherein the target subject data comprises an age and/or biological sex of the target subject, wherein the processing at least the location information comprises processing at least the location information and the age and/or biological sex of the target subject data to predict, for each of one or more diseases or conditions, the risk level.

5. The computer-implemented method of claim 4, wherein the step of processing the location information to predict a risk level for each of one or more diseases or conditions comprises, for each disease or condition:
obtaining a second disease/condition rate for other subjects in a same age group and/or biological sex as the target subject; and
using at least the second disease/condition rate to predict a risk level for the target subject to the said disease or condition.

6. The computer-implemented method of claim 5, wherein the step of processing the location information to predict a risk level for each of one or more diseases or conditions comprises, for each disease or condition:
combining, and optionally weighting, the first and second disease rates to produce the predicted risk level for the target subject to the said disease or condition.

7. The computer-implemented method of claim 1, further comprising obtaining genomic data of the target subject, comprising information on one or more genetic factors of the target subject,
wherein the processing at least the location information comprises processing at least the location information and the genomic data to predict, for each of one or more diseases or conditions, the risk level.

8. The computer-implemented method of claim 7, wherein the step of processing the location information to predict a risk level for each of one or more diseases or conditions comprises, for each disease or condition:
processing the genomic data to identify any genomic factors that influence a risk or rate of the disease or condition; and
using the identified genomic factors to predict a risk level for the target subject to the said disease or condition.

9. The computer-implemented method of claim 1, wherein the one or more diseases or conditions comprise only:
non-infectious diseases or conditions; and/or
long-term, degenerative and/or chronic diseases or conditions.

10. The computer-implemented method of claim 1, wherein the one or more treatment or diagnostic procedures comprises at least one preventative treatment procedure, such as at least one vaccination.

11. The computer-implemented method of claim 1, wherein the one or more treatment or diagnostic procedures comprises at least one disease screening procedure.

12. The computer-implemented method of claim 1, further comprising providing a visual representation of the recommended periodic healthcare plan.

13. A computer program product comprising computer program code means which, when executed on a computing device having a processing system, cause the processing system to perform all of the steps of the method according to claim 1.

14. A processing system configured to generate a recommended periodic healthcare plan for a target subject, wherein the recommended periodic healthcare plan indicates a recommended frequency for performing one or more treatment or diagnostic procedures on the target subject, the processing system being configured to:
obtain location information of the target subject, the location information indicating one or more historic locations of the target subject;
process at least the location information to predict, for each of one or more diseases or conditions, a risk level of the target subject to the said disease or condition; and
generate a recommended periodic healthcare plan including, for each of the one or more diseases or conditions, a recommended periodicity for performing a treatment or diagnostic procedure for said disease or condition, wherein the recommended periodicity is responsive to at least the risk level of the target subject.

15. A system comprising:
the processing system of claim 14; and
a user interface configured to display a visual representation of the recommended periodic healthcare plan.
